(19) United States
(12) Patent Application Publication
(10) Pub. No.: US 2009/0320852 A1
(43) Pub. Date: Dec. 31, 2009

(54) Tracheostomy Tube Butterfly Flange

(76) Inventors: Brian J. Cuevas, Cumming, GA (US); Joe Cesa, Cumming, GA (US); Michael Sleva, Atlanta, GA (US); Sam Chan, Atlanta, GA (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/147,952

(22) Filed: Jun. 27, 2008

(51) Int. Cl. A61M 16/00

(52) U.S. Cl. 128/207.14

(57) ABSTRACT

There is provided a novel tracheostomy tube flange. The flange is made of a flexible material and has a large open area for viewing of the underlying skin. The flange has holes for suturing the flange to the skin and slots for attachment of a strap to surround the neck and keep the flange and tube in place. The flange may swivel on the tube to allow for greater flexibility in attaching it to the skin. A stoma pad may be used with the flange to help keep the skin under the flange healthy.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through a stoma in the tracheal wall. Endotracheal tubes have been linked in some studies to an increased rate of ventilator acquired pneumonia (VAP) and so tracheostomy operations are becoming increasingly common and are being performed earlier in the patients hospital stay in order to reduce the occurrence of VAP.

After a tracheostomy procedure has been performed it is necessary to keep the tube in place. This is normally done by suturing a flange located on the proximal end of the tube to the skin of the patient. While this process has been used for some time it has not proven wholly satisfactory because patients may move or be moved and the inflexible flange can pull or damage the skin. It is also difficult to clean between the skin and flange using current flanges.

There remains a need for a flange that can more easily allow for cleaning and that can accommodate some patient movement without causing pain or damage. There is also a need for a pad that may be inserted under the flange to improve cleanliness and dryness of the skin.

### SUMMARY OF THE INVENTION

There is provided a novel tracheostomy tube flange. The flange is made of a flexible material and has a large open area for viewing of the underlying skin. The flange has holes for suturing the flange to the skin and slots for attachment of a strap to surround the neck and keep the flange and tube in place. The flange may swivel on the tube to allow for greater flexibility in attaching it to the skin. A stoma pad may be used with the flange to help keep the skin under the flange healthy.

### BRIEF DESCRIPTION OF THE DRAWINGS

FIG. 1 is a drawing of a tracheostomy tube 26 having a flange 70. (FIG. 18 of 5503b)

FIG. 2 is a drawing of the skin-facing side of the flange 70.

FIG. 3 is a drawing of the ventilator-facing side of the flange 70.

FIG. 4 is a drawing of a stoma pad 90 for use with the flange 70.

### DETAILED DESCRIPTION OF THE INVENTION

Tracheostomy is a lifesaving procedure to allow a patient to be ventilated directly through the trachea by installing a tracheostomy tube. Tracheostomy is also believed by many to prevent or retard the onset of ventilator acquired pneumonia (VAP). Once a tracheostomy is performed, the tube is held in place by a flange that is sutured to the skin of the patient. Tracheostomy tubes conventionally have a rigid flange that prevents or inhibits movement of the patient or causes injury or pain when the patient moves. It is difficult to clean the skin under the rigid flange and this can permit irritation and redness and/or infection to occur.

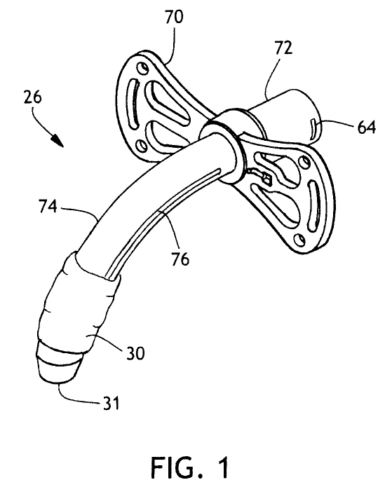

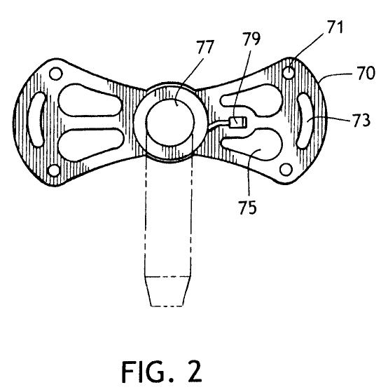

Turning to FIG. 1, there is a flange 70 on the trach tube 26 on the proximal end that is used to attach the trach tube to a patient's throat. The flange 70 extends on either side of the tube 26 near the proximal end where the ventilator connection 72 is located and has a central vent connector opening 77 (FIG. 2) through which the tube 26 extends. The flange 70 is flexible and non-irritating and can be sutured to the throat of a patient to anchor the tube 26. The flange 70 may desirably be of a width between 6 and 12 cm and height of 1 to 6 cm, more particularly between 7 and 10 cm and 2 and 5 cm respectively or still more particularly between 8 and 9 cm and 2 and 4 cm respectively. The size of the flange will vary depending on the size and needs of the patient. The flange is desirably made from polyurethane having a Duromoter ASTM D2240 Shore hardness of from about 50 D to 90 A though other suitable materials such as polyolefins and nylons may also be used.

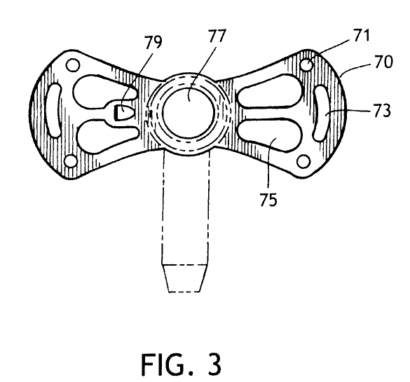

As shown in FIG. 1 the tube 26 also has a shaft 74 extending from the proximal end to the distal end. An inflation line 76 runs from the proximal end to the balloon cuff 30 so that the cuff may be inflated. As seen in FIGS. 2 and 3, the inflation line 79 continues through the flange 70 to a source of pressurizing gas (not shown).

The flange 70 is flexible and should be non-irritating to the skin. By being flexible the flange more easily conforms to the anatomy of a patient. The flange 70 may also be made so that it may swivel on the trach tube 26 so that it may be turned to a better position for suturing. The large open area 75 of the described flange allows for viewing of the area under the flange so that medical personnel may monitor the health of the skin and watch for infection or other complications. It is desired that the flange have an open area, not including the vent connector opening 77, of at least 20 percent, more desirably 30 percent and still more desirably at least 40 percent. There may be suturing points 71 desirably near the four corners of the flange 70. There may be two slots 73 for attachment of a strap (not shown) on the outer edges of the flange. Straps for securing the flange conventionally pass around the back of the neck of a patient to help to hold the tube 20 in place.

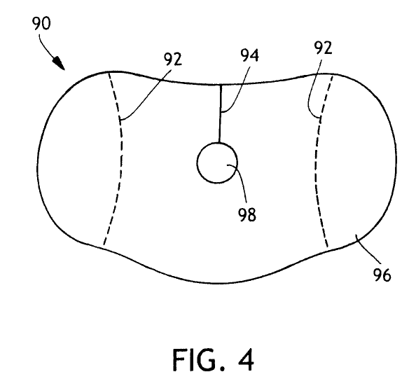

A stoma pad may be used with the flange 70 to absorb moisture or other exudates and help keep the skin clean and dry. The pad 90 is shown in FIG. 4 and has a central hole 98 that corresponds to the trach tube 26, a cut slit 94 that allows the pad to pass around the tube 26 and the edge may be bonded 96 to increase the structural integrity of the pad 90. The pad 90 desirably has two perforations 92 to allow sections of the pad to be removed if desired so as not to interfere with the optional securing straps that would pass through the slots 73. The pad may be somewhat larger than the flange, for example, 1 to 3 cm wider and higher and may be between 1 and 10 mm thick.

The stoma pad 90 may be made from a nonwoven material such as those made from spunbond, meltblown fibers as well as coform composites.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. Many differently polymers may be used to produce spunbond fibers including, for example, polyolefins like polyethylene and polypropylene.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are cared by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein, the term “coform” means a process in which at least one meltblown diehead is arranged near a chute through which other materials are added to the web while it is forming. Such other materials may be pulp, superabsorbent particles, natural polymers (for example, rayon or cotton fibers) and/or synthetic polymers (for example, polypropylene or polyester) fibers, for example, where the fibers may be of staple length. Coform processes are shown in commonly assigned U.S. Pat. No. 4,818,464 to Lau and U.S. Pat. No. 4,100,324 to Anderson et al. Webs produced by the coform process are generally referred to as coform materials.

As used herein “multilayer nonwoven laminate” means a laminate wherein some of the layers are spunbond and some meltblown such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown layers or multiple spunbond layers in many different configurations and may include other materials like films (F) or coform materials, e.g. SMMS, SM, SFS, etc.

One suitable stoma pad is made from a 150 gsm coform material made from a 70/30 mix of pulp and polypropylene with a facing layer on each side made from spunbond fibers. Other multilayer nonwoven laminates known to those skilled in the art may also be used.

In use the stoma pad 90 is opened at the cut slit 94 and slid up and under the flange 70. Removal of the outside portions of the pad at the perforations is up to the discretion of the caregiver.

This application is one of a group of commonly assigned patent application which are being filed on the same day. The group includes application Ser. No. ______ (attorney docket no. 64375503US01) in the name of Brian J. Cuevas and is entitled “Easy Grip Tapered Dilator”; application Ser. No. ______ (attorney docket no. 64375503US02) in the name of Brian J. Cuevas and is entitled “Method of Performing a Tracheostomy”; application Ser. No. ______ (attorney docket no. 64375504US01) in the name of Brian J. Cuevas and is entitled “Dilator Loading Catheter”; application Ser. No. ______ (attorney docket no. 64392563US01) in the name of Brian J. Cuevas and is entitled “Tracheostomy Tube Butterfly Flange”; application Ser. No. ______ (attorney docket no 64482359US01) in the name of James Schumacher and is entitled “Tracheostomy Tube”; design application Ser. No. ______ (attorney docket no. 64392563US02) in the name of Brian J. Cuevas and is entitled “Butterfly Flange”; design application Ser. No. ______ (attorney docket no. 64375503US03) in the name of Brian J. Cuevas and is entitled “Tapered Dilator Handle”; design application Ser. No. ______ (attorney docket no. 64392563US03) in the name of Brian J. Cuevas and is entitled “Stoma Pad”. The subject matter of these applications is hereby incorporated by reference.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A flange for use with a tracheostomy tube comprising a flexible polymer with a Durometer ASTM D2240 hardness between 50 D and 90.

2. The flange of claim 1 further comprising suturing points.

3. The flange of claim 2 having four suturing points located near corners of said flange.

4. The flange of claim 1 wherein said polymer is polyurethane.

5. The flange of claim 1 wherein said flange may swivel on said tracheostomy tube.

6. A flange on a tracheostomy tube comprising a stoma pad adapted to be placed between said flange and a patient's skin.

7. The flange of claim 6 wherein said stoma pad has perforations for removal of portions of said pad.

8. The flange of claim 7 wherein said pad is comprised of nonwoven fabric.

9. The flange of claim 8 wherein said pad is made from a coform material with spunbond facings on either side.

10. A flange for use with a tracheostomy tube comprising a flexible polymer and having an open area of at least 20 percent.

11. The flange of claim 10 wherein said open area is at least 30 percent.

12. The flange of claim 10 wherein said open area is at least 40 percent.
